(19) United States
(12) Patent Application Publication
(10) Pub. No.: US 2014/0262830 A1
(43) Pub. Date: Sep. 18, 2014

(54) METHOD FOR DETERMINING TEAR GLUCOSE CONCENTRATION WITH BLOOD GLUCOSE TEST STRIPS

(71) Applicant: The Regents of the University of Michigan, Ann Arbor, MI (US)

(72) Inventors: Kyoung Ha Cha, Ann Arbor, MI (US); Gary C. Jensen, Ann Arbor, MI (US); Bruce E. Cohan, Ann Arbor, MI (US); Mark E. Meyerhoff, Ann Arbor, MI (US)

(73) Assignee: The Regents of the University of Michigan, Ann Arbor, MI (US)

(21) Appl. No.: 14/208,891

(22) Filed: Mar. 13, 2014

Related U.S. Application Data
(60) Provisional application No. 61/779,575, filed on Mar. 13, 2013.

(51) Int. Cl. C12Q 1/00

(52) U.S. Cl. CPC C12Q 1/006
USPC 205/777.5

(57) ABSTRACT

A method for determining glucose concentration in tear fluid includes providing a blood glucose test strip having glucose dehydrogenase as an active enzyme provided therein, receiving a tear fluid sample via fluid communication of the blood glucose test strip with an eye region of a subject, and processing the tear fluid sample to determine a tear glucose concentration. The method may further include correlating the determined tear glucose concentration with a blood glucose concentration.

### CROSS-REFERENCE TO RELATED APPLICATIONS

This application claims the benefit of U.S. provisional application Ser. No. 61/779,575 filed Mar. 13, 2013, the disclosure of which is hereby incorporated in its entirety by reference herein.

### TECHNICAL FIELD

Embodiments relate to a method for determining tear glucose concentration using blood glucose test strips.

### BACKGROUND

Self-glucose monitoring technologies have drawn significant attention over the past several decades to help in the management of diabetes, which afflicts about 5% of the world population. Tight glycemic control is important to care for patients with diabetes to prevent long-term complications. In patients treated with insulin as in type I diabetes, glucose levels must be measured up to eight times a day to avoid the risk of hypoglycemia. These measurements require painful finger-pricking by the patient to obtain a blood sample for measurements using a strip-type glucometer.

A number of studies have been carried out to find a less invasive means to monitor blood glucose levels, including the use of infrared spectroscopy (Maruo K et al., Appl. Spectrosc., 2006, 60(12), 1423-1431; Mueller M et al., Sensor. Actuat. B-Chem., 2009, 142(2), 502-508), a GlucoWatch design that is based on electro-osmotic flow of subcutaneous fluid to surface of skin (Potts R O et al., Diabetes-Metab. Res. Rev., 2002, 18, S49-S53), and measurement of tissue metabolic heat conformation (Cho O K et al., Clin. Chem., 2004, 50(10), 1894-1898), but none of these techniques have yet yielded the quality of analytical results required to become widely used for blood glucose measurements.

### SUMMARY

In one embodiment, a method for determining glucose concentration in tear fluid includes providing a blood glucose test strip having glucose dehydrogenase as an active enzyme provided therein, receiving a tear fluid sample via fluid communication of the blood glucose test strip with an eye region of a subject, and processing the tear fluid sample to determine a tear glucose concentration.

In another embodiment, a method for determining glucose concentration in tear fluid includes providing a blood glucose test strip having glucose dehydrogenase as an active enzyme, pyrroloquinoline quinone as a coenzyme, and a nitrosoaniline derivative as an electron transfer mediator, receiving a tear fluid sample via fluid communication of the blood glucose test strip with an eye region of a subject, and processing the tear fluid sample to determine the glucose concentration in the tear fluid sample.

In another embodiment, a method for determining blood glucose concentration includes providing a blood glucose test strip having glucose dehydrogenase as an active enzyme provided therein, receiving a tear fluid sample having a volume of less than about 1 μL via fluid communication of the blood glucose test strip with an eye region of a subject, reacting the tear fluid sample with the active enzyme to determine the tear glucose concentration, and correlating the determined tear glucose concentration with a blood glucose concentration.

### BRIEF DESCRIPTION OF THE DRAWINGS

FIG. 1 shows calibrations plotted at the 1 minute mark for various commercial blood glucose test strips (N=5, 37° C.);

FIG. 2 depicts a calibration plot for Roche test strips at the 5 second mark;

FIG. 3 shows calibration plots for Roche and Nipro test strips at room temperature with 300 mV applied voltage;

FIG. 4 illustrates the calibration plots for Roche test strips at various applied voltages at the 5 second mark;

FIG. 5 illustrates the calibration plots for Nipro test strips at various applied voltages at the 5 second mark; and

FIG. 6 is a schematic illustration of a blood glucose test strip in fluid communication with a subject's eye region to obtain a tear fluid sample.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention. Except in the examples, or where otherwise expressly indicated, all numerical quantities in this description indicating amounts of material of conditions of reaction and/or use are to be understood as modified by the word “about” in describing the broadest scope of the invention. Practice within the numerical limits stated is generally preferred.

There have been a number of reports suggesting that tear glucose levels can be a monitor of blood glucose levels. However, accurate measurement of tear glucose concentrations is challenging owing to the low concentration of glucose present (5-500 μM) and the very small sample volume available (ca. 1 μL). Blood glucose glucometer devices are currently widely used and most are based on electrochemical detection in small volumes of blood (<1 μL) obtained from a finger prick.

The approach of testing glucose in tear fluid as a substitute for blood provides a non-invasive method of monitoring glucose concentration. If good correlation between blood glucose and tear glucose concentrations can be shown, measurement of tear glucose levels may provide an attractive indirect measurement method for blood glucose levels within the normal as well as hyperglycemic and hypoglycemic ranges. For such a method to be effective, tear fluid needs to be collected from a subject's eye using a non-stimulating method so that increases in tear production do not alter the naturally present glucose levels. At the same time, it is important to sample the tear fluid without perturbation of blood capillaries on the surface of the eye, which might result in tear samples with much higher levels of glucose than actually present in the neat tear fluid sample.

Although existing commercial electrochemical test strips are designed to measure blood glucose levels in the range of 3-20 mM, embodiments disclosed herein indicate that certain types of these strips can provide output currents that yield reproducible and linear amperometric responses to glucose in the 0-500 μM range as found in tear fluid. As shown in FIG. 6, a tear fluid sample may be received by fluid communication or engagement of a test strip T with a subject's eye region E, such as by a wicking action as is known in the art. In particular, glucometer test strips that employ glucose dehydrogenase (GDH) as the active enzyme may be suitable for measuring and monitoring tear glucose concentration. Even more particularly, strips manufactured by Roche Diagnostics, under the name of ACCU-CHEK® Aviva Plus, that utilize a pyrroloquinoline quinone (PQQ) coenzyme—GDH active enzyme and a nitrosoaniline derivative as the enzyme reaction mediator (see U.S. Pat. No. 7,727,467, incorporated by reference in its entirety herein) can be utilized according to the disclosed embodiments to measure and monitor tear glucose concentration in a small sample volume with acceptable accuracy and relatively low interference from both ascorbic and uric acid. In one non-limiting embodiment, the nitrosoaniline derivative may include o-methoxy-[N,N-bis-(2-hydroxyethyl)]-p-nitrosoaniline.

The blood glucose test strips may include adjunct materials to be used with the reagent composition, such as thickeners, viscosity modulators, film formers, stabilizers, buffers, detergents, gelling agents, fillers, film opening agents, coloring agents and thixotropic agents. Thickeners may include, for example cellulose and semi-synthetic cellulose derivatives such as, for example, carboxy-methylcellulose (CMC). Film forming and thixotropic agents may include polymers and silica such as, for example, polyvinylpyrrolidone (PVP). Additives may be utilized to control mass transport of potential interferences, but allow glucose to readily permeate the reagent layer, and thereby enhance the electrochemical selectivity of the test strip. For example, CMC will be anionic at neutral pH and could repel ascorbic acid and uric acid. Of course, additional adjunct materials, such as other anionic polymer additives, are also contemplated.

The sensitivity of PQQ-GDH test strips by Roche Diagnostics and Nipro Diagnostics were empirically validated by comparing them to other brands such as Bayer, Abbott, and Johnson & Johnson. Table 1 below lists all the brands and their specified enzymes (including flavin adenine dinucleotide (FAD)-GDH, nicotinamide adenine dinucleotide (NAD)-GDH, glucose oxidase (GOD) and PQQ-GDH)) and mediators. TRUEtest® strips by Nipro Diagnositcs were not included the first set of the experiments for the following comparison of sensitivity.

TABLE 1
Enzyme and mediator combinations used
in commercial glucometer test strips

| Company | Product | Enzyme | Mediator |
|---|---|---|---|
| Abbott | FreeStyle Lite ® | FAD-GDH | Osmium |
| Abbott | Precision Xtra ® | NAD-GDH | Phenanthroline |
| | | | Quinone |
| Bayer | CONTOUR ® | FAD-GDH | Potassium |
| | | | Ferricyanide |
| Johnson & | OneTouch ® Verio ™ | FAD-GDH | Potassium |
| Johnson | | | Ferricyanide |
| Johnson & | OneTouch ® Ultra ® | GOD | Potassium |
| Johnson | Blue | | Ferricyanide |
| Roche | ACCU-CHEK ® Aviva | PQQ-GDH | Nitosoaniline- |
| Diagnostics | Plus | | Derivative |
| Nipro | TRUEtest ® | PQQ-GDH | Unknown |
| Diagnostics | | | |

After wicking glucose solution (<1 μL) into each strip, the current was measured for 6 minutes at 400 mV applied voltage. A total of five strips were used for the measurement of the same glucose concentration to obtain reproducibility. Also, the experiment was conducted at physiological temperature, 37° C., to prepare for subsequent animal experiments. Based on the result, a calibration curve (0-100 μM) with the most linearity and lowest detection limit was found to be at the 1 minute mark after wicking in glucose solution (FIG. 1).

TABLE 2
Calculated detection limits for the tested brands

| | FreeStyle | Precision | | OneTouch ® | OneTouch ® | ACCU-CHEK ® |
|---|---|---|---|---|---|---|
| | Lite ® | Xtra ® | CONTOUR ® | Verio ™ | Ultra ® | Aviva Plus |
| Det. Limit | 140.9 | 612.9 | 86.0 | 112.3 | 286.08 | 13.5 |
| (μM) | | | | | | |

As shown in the calibrations of FIG. 1 and Table 2 above, the Roche ACCU-CHEK® Aviva Plus was the only strip that showed a reasonably low detection limit (less than about 15 μM), good linearity and reproducibility in the range of low end glucose concentrations. In addition, the Roche strip was the only strip which included gold electrodes, whereas other brands utilized carbon or palladium electrodes. In conclusion, test strips utilizing the PQQ-GDH enzyme with gold electrodes were found to be the most effective in measuring low end glucose concentrations compared to other brands.

For further optimization of calibration, a processing time mark at which Roche test strips should give the best calibration was found to be at the 5 second mark (FIG. 2) rather than at 1 minute mark based on the experimental result. The calibration detection limit at this time point is 8.68 μM (N=5).

Since Roche test strips, utilizing a PQQ-GDH enzyme, showed relatively better sensitivity as compared with other brands, Nipro Diagnostics test strips were tested as well, due to their utilization of PQQ-GDH enzyme and use of gold electrodes. Temperature-independency for glucose measurement of Roche and Nipro test strips were validated by performing the same procedure, but at room temperature with 300 mV applied voltage (FIG. 3). Detection limits for Roche and Nipro test strips are 3.0 μM and 17.0 μM, respectively (N=5). The calibrations shown in FIG. 3 provide evidence that Roche and Nipro test strips can measure low-end glucose concentration with significant reproducibility in a superior manner to other popular brands.

After obtaining calibration of the strips by Roche for glucose in buffer, three different concentrations of glucose (25, 50 and 75 μM), all with the highest amount of interferences (10 μM acetaminophen, 100 μM ascorbic acid, 100 μM uric acid), were tested at various applied voltages between 100-300 mV in order to test the strip's selectivity over interferences (N=3). The same experiment was conducted with Nipro Diagnostics test strips using the same interference mixture in three different glucose concentrations (25, 50 and 100 μM) (N=5). It has been reported in the literature that ascorbic and uric acid concentrations in tear fluid are ca. 20 and 70 μM, respectively (Choy C K M et al., Invest. Ophthalmol. Vis. Sci., 2000; Choy C K M et al., Optom. Vis. Sci., 2003). As a result, 100 μM of both ascorbic acid and uric acid were used to test the selectivity of the tear glucose sensor. For small neutral molecule interferences, 10 μM of acetaminophen was employed for testing, assuming that this species would be present in tear fluid at a similar relative dilution ratio compared to blood as glucose.

FIGS. 4 and 5 illustrate the calibrations for Roche and Nipro test strips (0-100 μM and 0-200 μM, respectively), at room temperature with different applied voltages and the results from testing the different glucose concentrations in interference solution. Tables 3 and 4 below provide data obtained with the Roche and Nipro test strips calibrated in pure glucose solution and then tested with standard glucose concentrations in interference solution at various applied voltages.

TABLE 3
Comparison of results for Roche test strips calibrated with standard
glucose solutions and tested for measuring standard glucose concentrations
in interference solution (10 μM acetaminophen, 100 μM ascorbic
acid, 100 μM uric acid) at various applied voltages

| Standard | | | | | | |
|---|---|---|---|---|---|---|
| [Glucose] in | Roche | | | | | |
| Interference | Calculated [Glucose] in Interference Solution (μM) | | | | | |
| Solution (μM) | 100 mV | 125 mV | 150 mV | 175 mV | 200 mV | 300 mV |
| 25 | 28.0 ± 0.3 | 25.7 ± 0.4 | 24.7 ± 1.0 | 23.5 ± 1.8 | 25.0 ± 1.2 | 21.4 ± 0.7 |
| 50 | 54.8 ± 2.7 | 50.5 ± 1.7 | 49.6 ± 0.8 | 48.1 ± 0.2 | 50.7 ± 3.4 | 47.2 ± 1.9 |
| 75 | 74.9 ± 1.7 | 70.1 ± 1.3 | 68.9 ± 1.6 | 71.2 ± 2.2 | 70.8 ± 2.4 | 69.2 ± 0.4 |

TABLE 4
Comparison of results for Nipro Diagnostics test strips calibrated with
standard glucose solutions and tested for measuring standard glucose
concentrations in interference solution (10 μM acetaminophen, 100
μM ascorbic acid, 100 μM uric acid) at various applied voltages

| Standard | | | | |
|---|---|---|---|---|
| [Glucose] in | Nipro Diagnostics | | | |
| Interference | Calculated [Glucose] in Interference Solution (μM) | | | |
| Solution (μM) | 50 mV | 100 mV | 200 mV | 300 mV |
| 25 | 211.21 ± 20.73 | 235.50 ± 4.09 | 179.69 ± 1.55 | 941.91 ± 140.02 |
| 50 | 150.38 ± 15.51 | 291.23 ± 35.72 | 189.18 ± 7.73 | 1049.99 ± 92.39 |
| 100 | 229.29 ± 14.21 | 361.26 ± 10.80 | 243.37 ± 8.73 | −190.31 |

As shown in the tables above, when using the Nipro test strips, large errors from the presence of interferences were observed at all applied voltages. However, with Roche strips, the errors are within a reasonable range of ≦14.5%. The percentage error for the calculated glucose concentrations in solutions containing interferences at different applied voltages using Roche test strips are presented in Table 5.

TABLE 5
Errors from the presence of interference in glucose solution
using Roche test strips at different applied voltages

| Standard | Roche | | | | | |
|---|---|---|---|---|---|---|
| [Glucose] in | Error for [Calculated Glucose] | | | | | |
| Interference | in Interference Solution (%) | | | | | |
| Solution (μM) | 100 mV | 125 mV | 150 mV | 175 mV | 200 mV | 300 mV |
| 25 | 11.8 | 2.9 | 1.1 | 6.0 | 0.1 | 14.5 |
| 50 | 9.6 | 1.0 | 0.8 | 3.7 | 1.5 | 5.6 |
| 75 | 0.2 | 6.6 | 8.1 | 5.1 | 5.6 | 7.7 |

TABLE 6
Comparison of limit of quantification at different
applied voltages using Roche test strips

| Roche | 100 mV | 125 mV | 150 mV | 175 mV | 200 mV | 300 mV |
|---|---|---|---|---|---|---|
| LOQ (μM) | 3.2 | 3.5 | 8.7 | 18.7 | 17.8 | 13.9 |

Optimal applied voltage, in terms of the best limit of quantitation (LOQ) and selectivity, for Roche strips can be identified based on the results presented in Tables 5 and 6. As shown in Table 5, all applied voltages show errors within a reasonable range, ≦14.5%, however, applied voltages of 150 mV and 200 mV exhibit lower % error than other applied voltages. As shown in Table 6, the LOQ for 150 mV and 200 mV applied voltages are 8.7 μM and 17.8 μM, respectively (N=3). However, three replicate measurements taken are insufficient to yield their “true” standard deviations and, in turn, their “true” LOQs. Thus, more measurements were tested for the new LOQs at 150 mV and 200 mV which resulted in values of 15.9 μM and 20.4 μM, respectively (N=9). From this result, the optimal applied voltage for Roche test strips can be empirically defined to be 150 mV because it yields the optimal selectivity, sensitivity and limit of quantification. This strip will have acceptable selectivity over major electroactive interferences found in tear fluid and results obtained for tear samples will likely reflect the true level of glucose present in such samples.

A blood glucometer device must be calibrated for each new batch of test strips. Roche test strips are labeled with a specific lot number, e.g. LOT #490702, and a designated code key that transfers its specific calibration into a glucometer once connected manually. Nipro diagnostics test strips employ a “no-coding” technology that allows the test strips to automatically calibrate and code the meter once inserted. Since it is important to match each new vial of test strips to a corresponding calibration, six different lots were tested for possible differences in their selectivity at the optimal applied voltage of 150 mV (N=3). The results are presented in Table 7 and the calculated errors from the presence of interference are reported in Table 8. Nipro diagnostics test strips were not tested in the same manner due to their poor selectivity over interferences (see Table 4, above).

TABLE 7
Comparison of selectivity results of six different
lots at 150 mV using Roche test strips

| Standard | Roche | | | | | |
|---|---|---|---|---|---|---|
| [Glucose] in | Calculated [Glucose] in Interference Solution (μM) | | | | | |
| Interference | Lot# | Lot# | Lot# | Lot# | Lot# | Lot# |
| Solution (μM) | 490702 | 490921 | 491302 | 491345 | 491610 | 491357 |
| 25 | 26.6 ± 1.1 | 23.9 ± 1.5 | 26.3 ± 0.8 | 25.9 ± 1.0 | 28.7 ± 1.0 | 24.7 ± 1.0 |
| 50 | 57.9 ± 1.7 | 50.6 ± 0.5 | 56.6 ± 4.1 | 53.0 ± 1.7 | 55.3 ± 1.6 | 49.6 ± 0.8 |
| 75 | 80.6 ± 3.3 | 75.3 ± 2.3 | 75.7 ± 1.7 | 75.0 ± 1.4 | 81.7 ± 2.6 | 68.9 ± 1.6 |

TABLE 8
Errors from the presence of interference in glucose solution
using different lot number of Roche test strips at 150 mV

| | Roche | | | | | |
|---|---|---|---|---|---|---|
| Standard | Error for [Calculated Glucose] | | | | | |
| [Glucose] in | in Interference Solution (%) | | | | | |
| Interference | Lot# | Lot# | Lot# | Lot# | Lot# | Lot# |
| Solution (μM) | 490702 | 490921 | 491302 | 491345 | 491610 | 491357 |
| 25 | 6.3 | 4.6 | 5.4 | 3.6 | 14.6 | 1.1 |
| 50 | 15.7 | 1.2 | 13.1 | 6.1 | 10.6 | 0.8 |
| 75 | 7.5 | 0.4 | 0.9 | 0.1 | 8.9 | 8.1 |

As presented in Tables 7 and 8, selectivity errors for six different lots are within a reasonable range of 15.7% using 150 mV applied voltage. However, since such differences in degree of errors do exist, one skilled in the art may be advised to select only a single lot for low-end tear glucose measurements and calibration for optimized and consistent results. Therefore, in terms of reproducibility, sensitivity and selectivity, blood glucose test strips can be used to precisely measure low-end glucose concentration in tear fluid at 150 mV applied voltage.

The invention is elucidated further by an example in the following.

### Example

### Human Tear Glucose Experiment

A potentiostat was used with the ACCU-CHEK® Aviva Plus glucometer test strips to amperometrically determine glucose in tear fluid sample (<1 μL) obtained from 3 fasting human subjects. Tear fluid was collected with 1 μL, microcapillaries. Calibration was obtained over the range of 0-100 μM using pure glucose solutions (150 mV applied potential, N=3, LOQ=3.8 μM). The range and median found for fasting tear glucose concentrations using the glucometer strips are 21.8-138.6 μM and 47.9 μM, respectively. In Table 9, the result is compared with the most recent study on tear glucose concentration using liquid chromatography with electrospray ionization mass spectrometry (Baca J T et al., S. A. Clin. Chem. 2007, 53, 1370-1372).

TABLE 9
Comparison of results by two different
groups on tear glucose concentration

| | Range | Median | # of | # of |
|---|---|---|---|---|
| | (μM) | (μM) | Subjects | Measurements |
| [Fasting Tear Glucose] | 21.8-138.6 | 47.9 | 3 | 54 |
| according to disclosed | | | | |
| Example | | | | |
| [Fasting Tear Glucose] | 7-161 | 28 | 25 | 148 |
| According to S. A. Clin. | | | | |
| Chem. publication | | | | |

Commercially available electrochemical test strips may be modified to provide an inlet for collecting microliter volumes of tear fluid for tear glucose measurements. For example, the distal tip of the strip may be manufactured to include a soft material suitable for contact with a subject's eye region, the width of the distal end of the strip may be tapered or otherwise altered to facilitate the collection of tear fluid, and/or the strip material may be treated to enhance its effectiveness and suitability for contact with the eye. The test strip may be modified to include polymeric layers which reduce or eliminate interferences from ascorbic acid and uric acid. For example, the strip can be modified by including one or more layers of NAFION® cation exchange polymer and an electropolymerized film of 1,3-diaminobenzene/resorcinol, so as to enhance the selectivity for glucose over potential known electroactive interferent species in tear fluid, including ascorbic acid and uric acid. In one embodiment, the glucometer test strip may be coated with a thin layer of NAFION® (e.g., ca. 5 μm thick). Then, electropolymerization of a solution containing 1.5 mM 1,3-diaminobenzene and a similar concentration of resorcinol in PBS buffer (0.1M, pH 7.4) may be initiated. NAFION® may be applied over the enzyme, such that glucose can reach the mediator but interferences cannot.

The requirements of tear glucose detection include a low detection limit (i.e., μM range), high selectivity over interferences such as ascorbic acid and uric acid, and the ability to measure small sample volumes as tear fluid can only be collected via a few microliters at a time. The blood glucose test strips may achieve very low detection limits of glucose that are required to monitor glucose levels in tear fluid. With this strip configuration, in one embodiment only about 1 μL of tear fluid is required in order to measure the glucose concentration.

It should be noted that this low detection limit may be achieved by not coating the outer surface of the sensor with an additional membrane that restricts diffusion of glucose to the enzymatic layer. Such an additional coating is typically required for blood and subcutaneous glucose sensing in order to ensure that oxygen is always present in excess compared to glucose in the enzymatic layer to achieve linear response to high glucose concentrations when detecting hydrogen peroxide as the product of the enzymatic reactions. However, given the much lower levels of glucose in tear fluid as compared to blood, and the fact that the selected strips will use GDH as the glucose selective enzyme (not glucose oxidase), no outer membrane is needed to retard glucose diffusion, and this ultimately enables the very low detection limit of the electrochemical test strips disclosed herein.

The glucometer test strips exhibit excellent selectivity over known electroactive interferences, a low detection limit, a wide dynamic range, excellent repeatability and in one embodiment requires less than about a 1 microliter sample volume. Use of tears as an alternate sample to assess blood glucose in human subjects may require that the ratio of glucose in tears and blood be established first for a given individual, so that the appropriate algorithm can be employed to report values that more closely reflect the true blood levels present.

In the potential real-world application of electrochemical test strips for monitoring diabetic patients, after a correlation between tear and blood glucose levels for each individual is established, an abnormal tear glucose concentration range may be set up to detect dangerous blood glucose levels from the correlation. Thus, tear glucose levels can be measured painlessly multiple times per day to monitor blood glucose level change without the pain from repeated invasive blood sampling. Blood glucose level can still be measured using the traditional blood collection method in order to trigger proper therapy when tear glucose detection suggests that blood glucose levels are out of the normal range.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method for determining glucose concentration in tear fluid, the method comprising:
providing a blood glucose test strip having glucose dehydrogenase as an active enzyme provided therein;
receiving a tear fluid sample via fluid communication of the blood glucose test strip with an eye region of a subject; and
processing the tear fluid sample to determine a tear glucose concentration.

2. The method of claim 1, wherein receiving the tear fluid sample includes obtaining a tear fluid sample of less than about 1 μL.

3. The method of claim 1, wherein processing the tear fluid sample includes a processing time of about 5 seconds.

4. The method of claim 1, wherein the blood glucose test strip includes pyrroloquinoline quinone as a coenzyme.

5. The method of claim 1, wherein the blood glucose test strip includes a nitrosoaniline derivative as an electron transfer mediator.

6. The method of claim 1, wherein the blood glucose test strip includes electrodes provided therein, and wherein processing the tear fluid sample includes applying a voltage to the electrodes to induce an electrochemical reaction of the active enzyme and the glucose in the tear fluid sample, and detecting a current produced by the electrochemical reaction from which the tear glucose concentration is determined.

7. The method of claim 6, wherein the electrodes include gold electrodes.

8. The method of claim 6, wherein the voltage applied to the electrodes is between about 150 mV and 200 mV.

9. The method of claim 1, wherein processing the tear fluid sample includes detecting glucose concentrations between about 0-500 μM in the tear fluid sample.

10. The method of claim 1, wherein providing a blood glucose test strip includes selecting a blood glucose test strip from a single lot of test strips.

11. The method of claim 1, further comprising correlating the determined tear glucose concentration with a blood glucose concentration.

12. A method for determining glucose concentration in tear fluid, the method comprising:
providing a blood glucose test strip having glucose dehydrogenase as an active enzyme, pyrroloquinoline quinone as a coenzyme, and a nitrosoaniline derivative as an electron transfer mediator;
receiving a tear fluid sample via fluid communication of the blood glucose test strip with an eye region of a subject; and
processing the tear fluid sample to determine the glucose concentration in the tear fluid sample.

13. The method of claim 12, wherein receiving the tear fluid sample includes obtaining a tear fluid sample of less than about 1μL.

14. The method of claim 12, wherein processing the tear fluid sample includes a processing time of about 5 seconds.

15. The method of claim 12, wherein the blood glucose test strip includes gold electrodes provided therein, and wherein processing the tear fluid sample includes applying a voltage to the electrodes to induce an electrochemical reaction of the active enzyme, coenzyme and electron transfer mediator with the glucose in the tear fluid sample, and detecting a current produced by the electrochemical reaction from which the tear glucose concentration is determined.

16. The method of claim 15, wherein the voltage applied to the electrodes is about 150 mV.

17. The method of claim 12, wherein processing the tear fluid sample includes detecting glucose concentrations less than about 15 μM.

18. The method of claim 12, wherein providing a blood glucose test strip includes selecting a glucose test strip from a single lot of test strips.

19. The method of claim 12, further comprising correlating the determined tear glucose concentration with a blood glucose concentration.

20. A method for determining blood glucose concentration, the method comprising:
providing a blood glucose test strip having glucose dehydrogenase as an active enzyme provided therein;
receiving a tear fluid sample having a volume of less than about 1 μL via fluid communication of the blood glucose test strip with an eye region of a subject;
reacting the tear fluid sample with the active enzyme to determine the tear glucose concentration; and
correlating the determined tear glucose concentration with a blood glucose concentration.
